(19) United States
(12) Patent Application Publication
(10) Pub. No.: US 2022/0265564 A1
(43) Pub. Date: Aug. 25, 2022

(54) CAPSID THERAPY

(71) Applicant: James Andrew Leskosek, Summerland (CA)

(72) Inventor: James Andrew Leskosek, Summerland (CA)

(21) Appl. No.: 17/675,931

(22) Filed: Feb. 18, 2022

Related U.S. Application Data
(60) Provisional application No. 63/152,101, filed on Feb. 22, 2021.

(51) Int. Cl. A61K 9/50

(52) U.S. Cl. CPC A61K 9/5068

(57) ABSTRACT

A lipid capsid comprising at least one S type protein for attachment to the ACE2 receptor of a cancer cell is provided. The lipid capsid encloses an inner package, comprising histone or a histone eviction compound. The S type protein of the lipid capsid attaches to the ACE2 receptor of a targeted cancer cell, enabling the outer lipid envelope of the capsid to be absorbed through the cell wall of the targeted cell, after which the inner package of the lipid capsid is delivered to the cancer cell. In some embodiments, the histone is selected from the H2AX histone, the H3K27 histone or the H3K9 histone, each histone having a different mechanism of action to disable the cancer cell or repair the DNA of the cancer cell. In another embodiment, the inner package contains a histone eviction compound for evicting histone from the cancer cell.

### FIELD

The present disclosure relates to using a capsid for delivery of therapeutic substances to a living cell for the treatment of cancer.

### BACKGROUND

There are many different therapies for cancer such as Radiation Therapy, Chemotherapy, Targeted Therapy, Hormone Therapy, Immunotherapy, and CRISPR therapy. Cancer is a disease that occurs when cells divide uncontrollably and spread into surrounding tissues. Cancer is caused by changes to DNA. Most cancer-causing DNA changes occur in sections of DNA called genes. It is thought that DNA methylation may be a primary mechanism that causes cancer in mammals.

### DESCRIPTION

In this new and innovative therapy, a capsid is made from lipids and S proteins. The S proteins are used for attachment to the ACE2 receptor of a cancer cell. Attachment to the ACE2 receptor allows the outer lipid envelope of the capsid to be absorbed into the cell wall, which then releases the inner package of the lipid capsid, contained within the outer lipid envelope, into the cell. In an embodiment, there are four different types of inner packages that may be delivered into the cell.

The first inner package would contain histone. The capsid would bombard the cell with histone. The histone would attach to the DNA strands that are open for transcription and other biomechanical functions. This would slow the targeted cell's progression and possibly stabilize the cell's natural chemical equilibrium. In another embodiment, histone H2AX variant may be delivered to the targeted cell and used for DNA repair.

The second inner package would contain histone H3K27, which may be used to repress chromatin transcription. The histone H3K27 tightens the DNA tighter around the histone in a cancer cell, thereby preventing transcription, slowing the cell's progression and possibly stabilizing the cell.

The third inner package would contain histone H3K9, which may be used to activate chromatin transcription. The histone H3K9 would be used to activate the DNA that may be otherwise repressed or silenced in a cancer cell.

The fourth inner package would be a histone eviction compound. This would evict the histone contained in the cancer cell for DNA repair machinery and transcription.

What is claimed is:

1. A lipid capsid comprising at least one S type protein for attachment to a cell wall of a cancer cell, the lipid capsid enclosing an inner package for delivery to the cancer cell, the inner package comprising histone.

2. The lipid capsid of claim 1, wherein the histone comprises histone H2AX for repairing DNA of the cancer cell.

3. The lipid capsid of claim 1, wherein the histone comprises histone H3K27 for repressing chromatin transcription of the cancer cell.

4. The lipid capsid of claim 1, wherein the histone comprises histone H3K9 for activating chromatin transcription of the cancer cell.

5. A lipid capsid comprising at least one S type protein for attachment to a cell wall of a cancer cell, the lipid capsid enclosing an inner package for delivery to the cancer cell, the inner package comprising a histone eviction compound for evicting the histone contained in the cancer cell.
